(19) United States
(12) Patent Application Publication
(10) Pub. No.: US 2023/0201600 A1
(43) Pub. Date: Jun. 29, 2023

(54) ELECTRICAL STIMULATION METHOD, DEVICE, AND COMPUTER-READABLE STORAGE MEDIUM FOR DETERMINING QUALITY OF ELECTRICAL-STIMULATION SIGNAL

(71) Applicant: GIMER MEDICAL. Co. LTD., New Taipei City (TW)

(72) Inventors: Wan-Ting CHIANG, New Taipei City (TW); Jian-Hao PAN, New Taipei City (TW)

(73) Assignee: GIMER MEDICAL. Co. LTD., New Taipei City (TW)

(21) Appl. No.: 17/974,920

(22) Filed: Oct. 27, 2022

(30) Foreign Application Priority Data
Dec. 29, 2021 (CN) 202111636422.4

(51) Int. Cl. A61N 1/36; A61N 1/02

(52) U.S. Cl. CPC A61N 1/3614; A61N 1/025

(57) ABSTRACT

An electrical stimulation method for determining the quality of an electrical-stimulation signal is provided in the invention. The electrical stimulation method is adapted for use in an electrical-stimulation device for performing an electrical stimulation. The electrical stimulation method may include the steps of generating the electrical-stimulation signal; sampling the electrical-stimulation signal; performing a fast Fourier transform on the sampled electrical-stimulation signal; and determining whether the signal quality of the electrical-stimulation signal on which the fast Fourier transform was performed meets a threshold.

### CROSS REFERENCE TO RELATED APPLICATIONS

This application claims priority of China Patent Application No. 202111636422.4, filed on Dec. 29, 2021, the entirety of which is incorporated by reference herein.

### BACKGROUND

### Technology Field

The embodiments of present invention mainly relate to an electrical stimulation technique.

### Description of the Related Art

In recent years, several therapeutic electrical nerve stimulation devices have been developed, and tens of thousands of people receive surgery for the implantation of electrical-stimulation devices each year. With the development of precision manufacturing techniques, medical instruments (such as implantable electrical-stimulation devices) have been miniaturized and may be implanted into the human body.

When the electrical-stimulation device is performing electrical stimulation, the quality of the electrical-stimulation signal generated by the electrical-stimulation device can affect the efficacy of the electrical stimulation. Therefore, how to determine the quality of the electrical-stimulation signal is an important issue.

### SUMMARY

In view of the issues of the prior art addressed above, an embodiment of the present disclosure provides a method, an electrical-stimulation device, and a computer-readable storage medium for determining the quality of an electrical-stimulation signal.

An embodiment of the present disclosure provides an electrical stimulation method for determining the quality of an electrical-stimulation signal. The electrical stimulation method may be adapted for use in an electrical-stimulation device for performing an electrical stimulation. The steps of the electrical stimulation method for determining the quality of an electrical-stimulation signal are as follows: generating the electrical-stimulation signal; sampling the electrical-stimulation signal; performing a fast Fourier transform on the sampled electrical-stimulation signal; and determining whether the signal quality of the electrical-stimulation signal on which the fast Fourier transform was performed meets a threshold.

An embodiment of the present disclosure provides an electrical-stimulation device. The electrical-stimulation device is configured to perform electrical stimulation. The electrical-stimulation device includes an electrical-stimulation signal-generating circuit, a sampling module, a fast Fourier transform calculation module, and a determination module. The electrical-stimulation signal-generating circuit is configured to generate an electrical-stimulation signal. The sampling module is configured to sample the electrical-stimulation signal. The fast Fourier transform calculation module is configured to perform a fast Fourier transform on the sampled electrical-stimulation signal. The determination module is configured to determine whether the signal quality of the sampled electrical-stimulation signal on which the fast Fourier transform was performed meets a threshold.

An embodiment of the present disclosure provides a computer-readable storage medium. The computer-readable storage medium stores one or more instructions and cooperates with an electrical-stimulation device for performing an electrical stimulation. When the instructions are executed by the electrical-stimulation device, the electrical-stimulation device executes a plurality of steps including generating an electrical-stimulation signal; sampling the electrical-stimulation signal; performing a fast Fourier transform on the sampled electrical-stimulation signal; and determining whether the signal quality of the electrical-stimulation signal on which the fast Fourier transform was performed meets a threshold.

Persons skilled in the art may achieve additional features and advantages of the present disclosure by modifying and altering the electrical stimulation method for impedance compensation and the electrical stimulation system disclosed in the detailed description of the present disclosure without departing from the spirit and scope of the present invention.

### BRIEF DESCRIPTION OF DRAWINGS

The invention can be more fully understood by reading the subsequent detailed description and examples with references made to the accompanying drawings, wherein:

FIG. 1 is a block diagram of an electrical-stimulation device 100 according to an embodiment of the present invention;

FIG. 2A is a schematic diagram of the electrical-stimulation device 100 according to an embodiment of the present invention;

FIG. 2B is a schematic diagram of the electrical-stimulation device 100 according to another embodiment of the present invention;

FIG. 3 is a waveform diagram of an electrical-stimulation signal of the electrical-stimulation device according to an embodiment of the present invention;

FIG. 4 is a detailed schematic diagram of the electrical-stimulation device 100 according to an embodiment of the present invention;

FIG. 5A shows a first set of predetermined target energy values according to an embodiment of the present invention;

FIG. 5B shows a second set of predetermined target energy values according to another embodiment of the present invention;

FIG. 6 is a block diagram of a control unit 140 according to an embodiment of the present invention;

FIG. 7 is a block diagram of an impedance compensation device 700 according to an embodiment of the present invention;

FIG. 8A is a schematic diagram of an impedance compensation model according to an embodiment of the present invention;

FIG. 8B is a schematic diagram of an impedance compensation model according to another embodiment of the present invention;

FIG. 9 shows a flow chart 900 of an electrical stimulation method for determining the quality of an electrical-stimulation signal according to an embodiment of the present invention; and

FIG. 10 shows a flow chart 1000 of an electrical stimulation method for determining the quality of an electrical-stimulation signal according to another embodiment of the present invention.

### DETAILED DESCRIPTION OF THE INVENTION

The following description explains preferred implementations of the present invention, which is made for the purpose of illustrating the general principles of the invention and should not be taken in a limiting sense. The scope of the invention is best determined by reference to the appended claims.

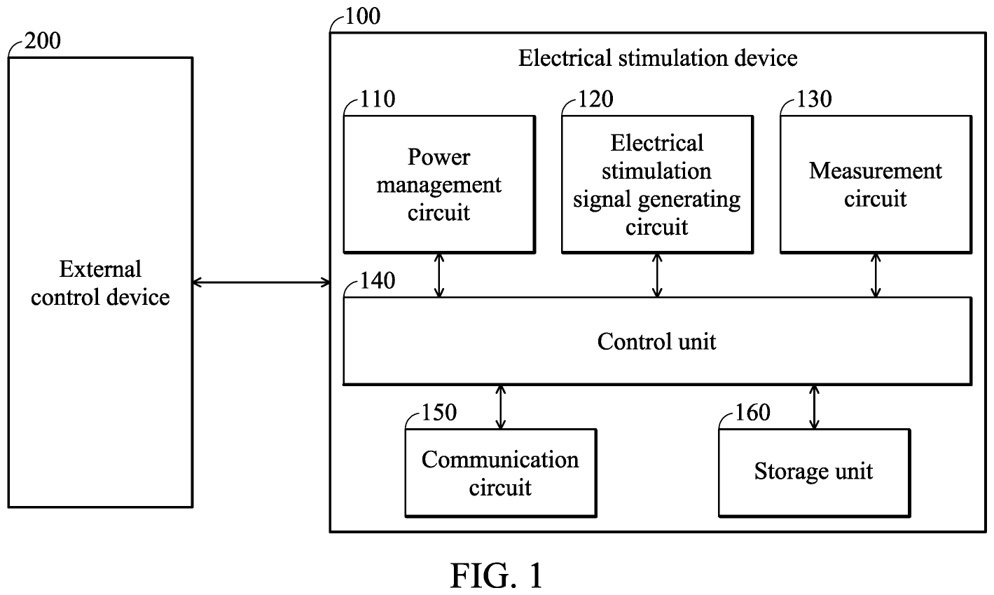

FIG. 1 is a block diagram of an electrical-stimulation device 100 according to an embodiment of the present invention. As shown in FIG. 1, the electrical-stimulation device 100 may include at least a power management circuit 110, an electrical-stimulation signal-generating circuit 120, a measurement circuit 130, a control unit 140, a communication circuit 150 and a storage unit 160. Note that the block diagram shown in FIG. 1 is merely illustrated for the convenience of explaining the embodiments of the present invention, but the invention is not limited thereto. The electrical-stimulation device 100 may include other elements.

According to an embodiment of the present invention, the electrical-stimulation device 100 may be electrically coupled to an external control device 200. The external control device 200 may have an operation interface. According to the input by a user via the operation interface, the external control device 200 may generate a command or signal to send to the electrical-stimulation device 100, and it may then send that command or signal to the electrical-stimulation device 100 via wired communication (e.g. a transmission line).

Additionally, according to another embodiment of the present invention, the external control device 200 may send the command or signal to the electrical-stimulation device 100 by wireless communication, such as Bluetooth, Wi-Fi or near field communication (NFC).

According to an embodiment of the present invention, the electrical-stimulation device 100 may be an implantable electrical-stimulation device, an external electrical-stimulation device with a lead implanted into a human body, or a transcutaneous electrical nerve stimulation device (TENS). According to an embodiment of the present invention, when the electrical-stimulation device 100 is a non-implantable electrical-stimulation device (e.g. an external electrical-stimulation device or a transcutaneous electrical-stimulation device), the electrical-stimulation device 100 and the external control device 200 may be integrated as a single device. According to an embodiment of the present invention, the electrical-stimulation device 100 may be an electrical-stimulation device with a battery, or an electrical-stimulation device with power wirelessly transmitted from the external control device 200. According to an embodiment of the present invention, in a trial phase, the electrical-stimulation device 100 is an external electrical-stimulation device with a lead implanted into a human body, the lead has electrodes thereon, and the external electrical-stimulation device sends an electrical-stimulation signal to a corresponding target region through the electrodes on the lead. In the trial phase, after the end of the lead with the electrode is implanted into the human body, the other end of the lead is connected with the external control device 200, and the external electrical-stimulation device may send an electrical-stimulation signal to evaluate whether the treatment is effective, and to check whether the lead functions properly and whether the position of lead implantation is correct. In the trial phase, the external control device 200 first pairs wirelessly with the external electrical-stimulation device (i.e. the non-implantable electrical-stimulation device). After the lead is implanted into the human body, the external electrical-stimulation device (i.e. the non-implantable electrical-stimulation device) is connected to the lead, and the external control device 200 wirelessly controls the external electrical-stimulation device (i.e. the non-implantable electrical-stimulation device) to perform electrical stimulation of the human body. According to an embodiment of the present invention, if the treatment is evaluated as effective in the trial phase, a permanent implantation phase may be performed. In the permanent implantation phase, the electrical-stimulation device 100 may be implanted into the human body with the lead, and the electrical-stimulation device 100 sends the electrical-stimulation signal to the corresponding target region through the electrodes on the lead. When the external control device 200 is entering the permanent implantation phase, the user or a medical doctor first needs to tap a phase change card to the external control device 200 to change the using status of the external control device 200 from the trial phase to the permanent implantation phase through NFC, and the external control device 200 may select an upper-limit target energy value and a lower-limit target energy value from a first set of target energy values according to a given electrical stimulation level. Subsequently, the external control device 200 may generate a second set of target energy values according to the upper-limit target energy value and the lower-limit target energy value (which is further described below). In addition, before the permanent implantation surgery or in the permanent implantation phase, the external control device 200 and the implantable electrical-stimulation device are wirelessly paired, the external electrical-stimulation device (i.e. the non-implantable electrical-stimulation device) is removed, and the electrical-stimulation device 100 (i.e. the implantable electrical-stimulation device) is connected with the lead and implanted into the human body.

According to an embodiment of the present invention, the power management circuit 110 is configured to provide power to the elements and circuits within the electrical-stimulation device 100. The power provided by the power management circuit 110 may come from a built-in rechargeable battery or the external control device 200, but the invention is not limited thereto. The external control device 200 may provide power to the power management circuit 110 by a wireless power-providing technique. The power management circuit 110 may be enabled or disabled according to a command from the external control device 200. According to an embodiment of the present invention, the power management circuit 110 may include a switch circuit (not shown in the figure). The switch circuit may be turned on or off according to the command from the external control device 200, so as to enable or disable the power management circuit 110.

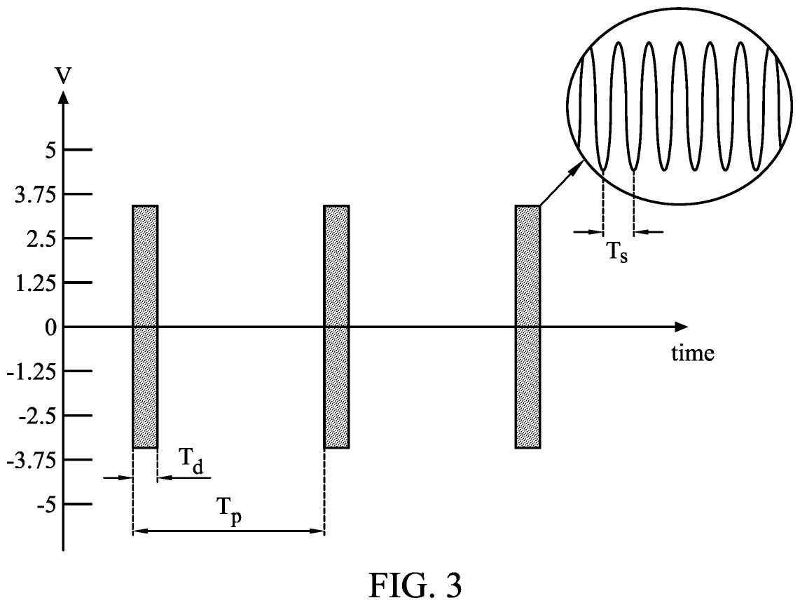

According to an embodiment of the present invention, the electrical-stimulation signal-generating circuit 120 is configured to generate an electrical-stimulation signal. The electrical-stimulation device 100 may send the electrical-stimulation signal to the electrodes on the lead through at least one lead, so as to perform electrical stimulation of a target region in the body of a user (a human or an animal) or a patient. Examples of possible target regions include a spinal cord, a spinal nerve, a dorsal root ganglia (DRG), a cranial nerve, a vagus nerve, a trigeminal nerve, a lateral recess, and a peripheral nerve, but the invention is not limited thereto. The structure of the electrical stimulation generating circuit 120 is explained in more detail in FIG. 4.

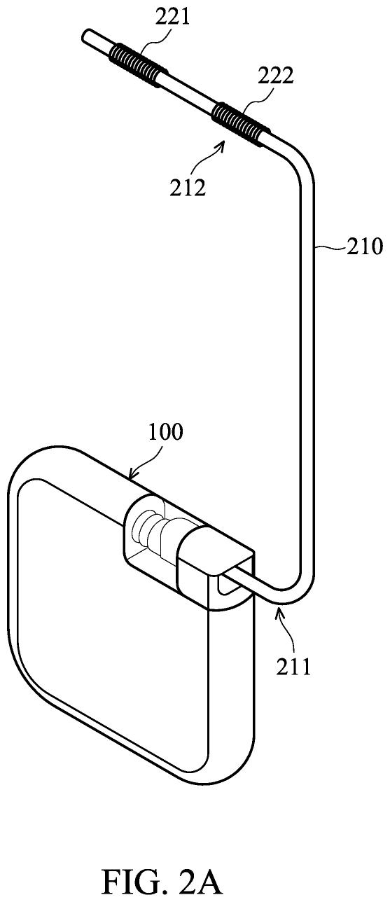

FIG. 2A is a schematic diagram of the electrical-stimulation device 100 according to an embodiment of the present invention. As shown in FIG. 2A, an electrical-stimulation signal may be output to a lead 210 to allow the electrical-stimulation signal to be sent from an end 211 of the lead 210 to another end 212 of the lead 210 (an electrode 221 or 222), so as to perform an electrical stimulation operation in a target region. In an embodiment of the present invention, the electrical-stimulation device 100 and the lead 210 are electrically connected in a detachable manner, but the invention is not limited thereto. For example, the electrical-stimulation device 100 and the lead 210 may be an integrally formed device.

FIG. 2B is a schematic diagram of the electrical-stimulation device 100 according to another embodiment of the present invention. As shown in FIG. 2B, electrodes 321 and 322 may be directly formed on a side of the electrical-stimulation device 100. The electrical-stimulation signal may be transmitted to the electrode 321 or the electrode 322 for performing an electrical stimulation operation at a target region. That is, in this embodiment, the electrical-stimulation device 100 does not need a lead to send the electrical-stimulation signal to the electrodes 321 and 322.

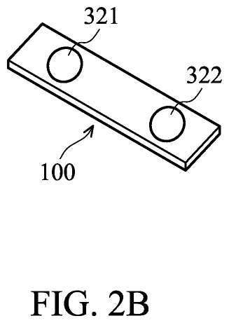

FIG. 3 is a waveform diagram of an electrical-stimulation signal of the electrical-stimulation device according to an embodiment of the present invention. As shown in FIG. 3, according to an embodiment of the present invention, the electrical-stimulation signal may be a pulsed radio-frequency (PRF) signal (also referred to as a pulsed signal), a continuous sine wave, a continuous triangular form or the like, but the present invention is not limited thereto. Additionally, when the electrical-stimulation signal is a pulsed alternating signal, a pulse cycle time Tₚ includes a pulsed signal and at least one resting period, and the pulse cycle time Tₚ is the reciprocal of a pulse repetition frequency. The range of the pulse repetition frequency (also referred to as the pulse frequency range) is, for example, between 0 and 1 KHz, preferably between 1 and 100 Hz, and the pulse repetition frequency of the electrical-stimulation signal in the present embodiment is, for example, 2 Hz. Furthermore, a duration time T_{d} (i.e. a pulse width) of a pulse in a pulse cycle time is between, for example, 1 and 250 ms (milliseconds), preferably between 10 and 100 ms, and the duration time T_{d} of the present embodiment is described as 25 ms as an example. In the present embodiment, the frequency of the electrical-stimulation signal is 500 KHz. In other words, the cycle time Tₛ of the electrical-stimulation signal is approximately 2 microseconds (μs). Additionally, the frequency of the electrical-stimulation signal is the intra-pulse frequency within each pulsed alternating signal shown in FIG. 3. In some embodiments, the range of the intra-pulse frequency of the electrical-stimulation signal is between, for example, 1 KHz and 1000 KHz. Note that in the embodiments of the present invention, if merely a “frequency” of the electrical-stimulation signal is described, it refers to the intra-pulse frequency of the electrical stimulation frequency. Furthermore, the range of the intra-pulse frequency of the electrical stimulation frequency is, for example, between 200 KHz and 800 KHz. Moreover, the range of the intra-pulse frequency of the electrical stimulation frequency is, for example, between 480 KHz and 520 KHz. Furthermore, the intra-pulse frequency of the electrical stimulation frequency is, for example, 500 KHz. The voltage range of the electrical-stimulation signal may be between −25V and +25V. Furthermore, the voltage range of the electrical-stimulation signal may be further between −20V and +20V. The current range of the electrical-stimulation signal may be between 0 and 60 mA. Furthermore, the current range of the electrical-stimulation signal may be further between 0 and 50 mA.

According to an embodiment of the present invention, the user may operate the electrical-stimulation device 100 to perform electrical stimulation when needed (for example, when a symptom worsens or not relieved). After the electrical-stimulation device 100 performs an electrical stimulation to the target region, the electrical-stimulation device 100 needs to wait for a limit time before performing the next electrical stimulation to the target region. For example, after the electrical-stimulation device 100 performs an electrical stimulation, the electrical-stimulation device 100 needs to wait for 30 minutes (i.e. the limit time) before performing the next electrical stimulation to the target region, but the present invention is not limited thereto. The limit time may be any time duration within 45 minutes, 1 hour, 4 hours or 24 hours.

According to an embodiment of the present invention, the measurement circuit 130 may measure the voltage and current of the electrical-stimulation signal according to the electrical stimulation generated by the electrical-stimulation signal-generating circuit 120. Additionally, the measurement circuit 130 may measure the voltage and current of the tissue in a target region in the body of the user or patient. According to an embodiment of the present invention, the measurement circuit 130 may adjust the current and the voltage of the electrical-stimulation signal according to an instruction from the control unit 140. The structure of the measurement circuit 130 is explained in more detail below with reference to FIG. 4.

According to an embodiment of the present invention, the control unit 140 may be a controller, a microcontroller or a processor, but the present invention is not limited thereto. The control unit 140 may be configured to control the electrical-stimulation signal-generating circuit 120 and the measurement circuit 130. The operation of the control unit 140 is explained below referring to FIG. 4.

According to an embodiment of the present invention, the communication circuit 150 may be configured to communicate with the external control device 200. The communication circuit 150 may send a command or signal received from the external control device 200 to the control unit 140, and send data measured by the electrical-stimulation device 100 to the external control device 200. According to an embodiment of the present invention, the communication circuit 150 may communicate with the external control device 200 via wireless or wired communication.

According to an embodiment of the present invention, when performing electrical stimulation, all the electrodes of the electrical-stimulation device 100 are activated (or enabled). Thus, the user does not need to select which of the electrodes on the lead are to be activated, and does not need to select which of the activated electrodes are negative or positive in polarities. For example, if the electrical-stimulation device 100 is equipped with eight electrodes, then the eight electrodes may be four positive electrodes and four negative electrodes arranged alternately.

Compared to a conventional electrical stimulation, which is a low-frequency (e.g. 10 KHz) pulsed signal prone to cause a tingling pain or paresthesia of the user, resulting in a discomfort of the user, in an embodiment of the present invention, the electrical stimulation is a high-frequency (e.g. 500 KHz) pulsed signal, which causes no or little paresthesia of the user.

According to an embodiment of the present invention, the storage unit 160 may be a volatile memory (e.g. a random access memory (RAM)), a non-volatile memory (e.g. a flash memory), a read-only memory (ROM), a hard disk drive, or a combination thereof. The storage unit 160 may be configured to store a file and data required for performing an electrical stimulation. According to an embodiment of the present invention, the storage unit 160 may be configured to store information related to a look-up table provided by the external control device.

FIG. 4 is a schematic diagram of the electrical-stimulation device 100 according to an embodiment of the present invention. As shown in FIG. 4, the electrical-stimulation signal-generating circuit 120 may include a variable resistor 121, a waveform generator 122, a differential amplifier 123, a channel switch circuit 124, a first resistor 125 and a second resistor 126. The measurement circuit 130 may include a current measurement circuit 131 and a voltage measurement circuit 132. Note that the schematic diagram shown in FIG. 4 is illustrated merely for the convenience of explaining an embodiment of the present invention, but the present invention is not limited thereto. The electrical-stimulation device 100 may include additional elements, or include additional equivalent circuits.

As shown in FIG. 4, according to an embodiment of the present invention, the variable resistor 121 may be coupled to a serial peripheral interface (SPI) (not shown in the figure) of the control unit 140. The control unit 140 may send a command to the variable resistor 121 through the SPI to adjust the resistance of the variable resistor 121, so as to adjust the magnitude of the electrical-stimulation signal to output. The waveform generator 122 may be coupled to a pulse width modulation (PWM) signal generator (not shown in the figure) of the control unit 140. The PWM signal generator may generate a square wave and send the square wave to the waveform generator 122. After receiving the square wave generated by the PWM signal generator, the waveform generator 122 transforms the square wave to a sine wave signal and sends the sine wave signal to the differential amplifier 123. The differential amplifier 123 may transform the sine wave signal to a differential signal (i.e. the electrical-stimulation signal to output) and sends the differential signal to the channel switch circuit 124 through the first resistor 125 and the second resistor 126. The channel switch circuit 124 may sequentially send the differential signal (i.e. the electrical-stimulation signal to output) to the electrode corresponding to each channel through a lead L according to the command from the control unit 140.

As shown in FIG. 4, according to an embodiment of the present invention, the current measurement circuit 131 and the voltage measurement circuit 132 may be coupled to the differential amplifier 123 to obtain the current and voltage of the differential signal (i.e. the electrical-stimulation signal to output). Additionally, the current measurement circuit 131 and the voltage measurement circuit 132 may be configured to measure the voltage and current of the tissue in a target region in the body of a user or patient. In addition, the current measurement circuit 131 and the voltage measurement circuit 132 may be coupled to the input/output (I/O) interface of the control unit (not shown in the figure) to receive a command from the control unit 140. According to the command from the control unit 140, the current measurement circuit 131 and the voltage measurement circuit 132 may adjust the current and the voltage of the electrical-stimulation signal to a current and a voltage that are more suitable for the control unit 140 to process. For example, if the voltage measured by the voltage measurement circuit 132 is ±10V and the voltage that is more suitable for the control unit 140 to process is 0 to 3V, then the voltage measurement circuit 132 may scale down the voltage to ±1.5V and then shift the voltage to 0-3V according to the command from the control unit 140.

After the current measurement circuit 131 and the voltage measurement circuit 132 have adjusted the current and the voltage, the current measurement circuit 131 and the voltage measurement circuit 132 send the adjusted electrical-stimulation signal to an analog-to-digital converter (ADC) (not shown in the figure) of the control unit 140. The ADC samples the electrical-stimulation signal for subsequent computation and analysis by the control unit 140.

According to an embodiment of the present invention, when an electrical stimulation in a target region in the body of a patient is desired, a user (may be medical personnel or the patient himself/herself) may select an electrical stimulation level from multiple electrical stimulation levels on an operation interface of the external control device 200. In an embodiment of the present invention, different electrical stimulation levels may correspond to different target energy values. The target energy values may be a set of predetermined energy values. When the user selects an electrical stimulation level, the electrical-stimulation device 100 may be informed of the energy value (in millijoules) to provide to the target region to perform the electrical stimulation according to the target energy of the electrical stimulation level selected by the medical doctor or the user. According to an embodiment of the present invention, in the trial phase, a plurality of target energy of a plurality of electrical stimulation levels may be regarded as a first set of predetermined target energy values. According to an embodiment of the present invention, the first set of predetermined target energy values (i.e. the plurality of target energy values) may be a linear sequence, an arithmetic sequence or a geometric sequence, but the present invention is not limited thereto.

According to an embodiment of the present invention, in the trial phase, the external control device 200 may have a first look-up table. In this embodiment, each electrical stimulation level and its corresponding target energy value may be recorded in the first look-up table. Thus, according to the electrical stimulation level selected by the user, the external control device 200 may search the first look-up table and obtain the target energy of the electrical stimulation level selected by the user from the first set of target energy values. After obtaining the target energy of the electrical stimulation level selected by the user, the external control device 200 sends the target energy value to the electrical-stimulation device 100. Then the electrical-stimulation device 100 may perform the electrical stimulation to the target region according to the target energy value.

According to another embodiment of the present invention, the electrical-stimulation device 100 may have a built-in first look-up table (e.g. a first look-up table stored in in the storage unit 160). In this embodiment, each electrical stimulation level and its corresponding target energy value may be recorded in the first look-up table. When the user selects an electrical stimulation level from the external control device 200, the external control device 200 sends a command to inform the control unit 140 of the electrical-stimulation device 100 of the electrical stimulation level selected by the user. Then, the control unit 140 may select the target energy of the electrical stimulation level selected by the user from the first set of target energy values according to the built-in first look-up table. After obtaining the target energy value, the electrical-stimulation device 100 may perform the electrical stimulation to the target region according to the selected target energy value until the corresponding first target energy value is sent to the target region, then stop the electrical stimulation to finish an electrical stimulation treatment.

According to another embodiment of the present invention, the communication circuit 150 may first obtain the electrical stimulation level selected by the user and the first look-up table from the external control device 200. In this embodiment, each electrical stimulation level and its corresponding target energy value may be recorded in the first look-up table. Then, the control unit 140 selects the target energy of the electrical stimulation level selected by the user from the first set of target energy values according to the electrical stimulation level selected by the user and the first look-up table obtained from the external control device 200. After obtaining the target energy value, the electrical-stimulation device 100 may perform the electrical stimulation to the target region according to the selected target energy value.

According to an embodiment of the present invention, the user may start by selecting the lowest electrical stimulation level (corresponding to the lowest target energy value in the first set of target energy values) and may then select the next target energy value in the first set of target energy values when the time limit on the electrical stimulation has elapsed. When the user finds a preferred or effective target energy value in electrical stimulation, such target energy value may be regarded as a given target energy value, and the electrical stimulation level of the given target energy value may be regarded as a given electrical stimulation level.

According to an embodiment of the present invention, in the permanent implantation phase, the external control device 200 (e.g. a controller of the external control device 200) may select an upper-limit target energy value and a lower-limit target energy value from the first set of target energy values according to the given electrical stimulation level. Then, the external control device 200 (e.g. a controller of the external control device 200) may generate a second set of target energy values according to the upper-limit target energy value and the lower-limit target energy value. In this embodiment, the external control device 200 (e.g. a controller of the external control device 200) generates a second look-up table according to the electrical stimulation level of each target energy value in the second set of target energy values. The external control device 200 sends the second look-up table or related parameter information to the electrical-stimulation device 100. When the user operates the external control device 200, the electrical-stimulation device 100 performs the electrical stimulation operation according to the second look-up table or the related parameter information. According to an embodiment of the present invention, in the trial phase, the electrical stimulation is performed to human body by an external electrical-stimulation device (i.e. a non-implantable electrical-stimulation device) according to a first set of target energy values selected by the user in the first look-up table; in the permanent implantation phase, the electrical stimulation is performed to human body by the electrical-stimulation device 100 (i.e. an implantable electrical-stimulation device) according to a second set of target energy values selected by the user in the second look-up table. In an embodiment of the present invention, the electrical-stimulation device 100 performs the electrical stimulation to the target region until the corresponding second target energy value is sent to the target region, then stop the electrical stimulation to finish an electrical stimulation treatment.

According to another embodiment of the present invention, in the permanent implantation phase, the electrical-stimulation device 100 may select an upper-limit target energy value and a lower-limit target energy value from the first set of target energy values according to the given electrical stimulation level. Then, the electrical-stimulation device 100 may generate a second set of target energy values according to the upper-limit target energy value and the lower-limit target energy value. In this embodiment, the electrical-stimulation device 100 generates a second look-up table according to the electrical stimulation level of each target energy value in the second set of target energy values. The electrical-stimulation device 100 sends the second look-up table or related parameter information to the external control device 200. When the user operates the external control device 200, the electrical-stimulation device 100 performs the electrical stimulation operation according to the second look-up table or the related parameter information.

According to an embodiment of the present invention, the second set of target energy values may be a linear sequence, an arithmetic sequence or a geometric sequence, but the present invention is not limited thereto. According to an embodiment of the present invention, the number of target energy values included in the first set of target energy values may be equal to the number of target energy values included in the second set of target energy values. According to another embodiment of the present invention, the number of target energy values included in the first set of target energy values may be different from the number of target energy values included in the second set of target energy values.

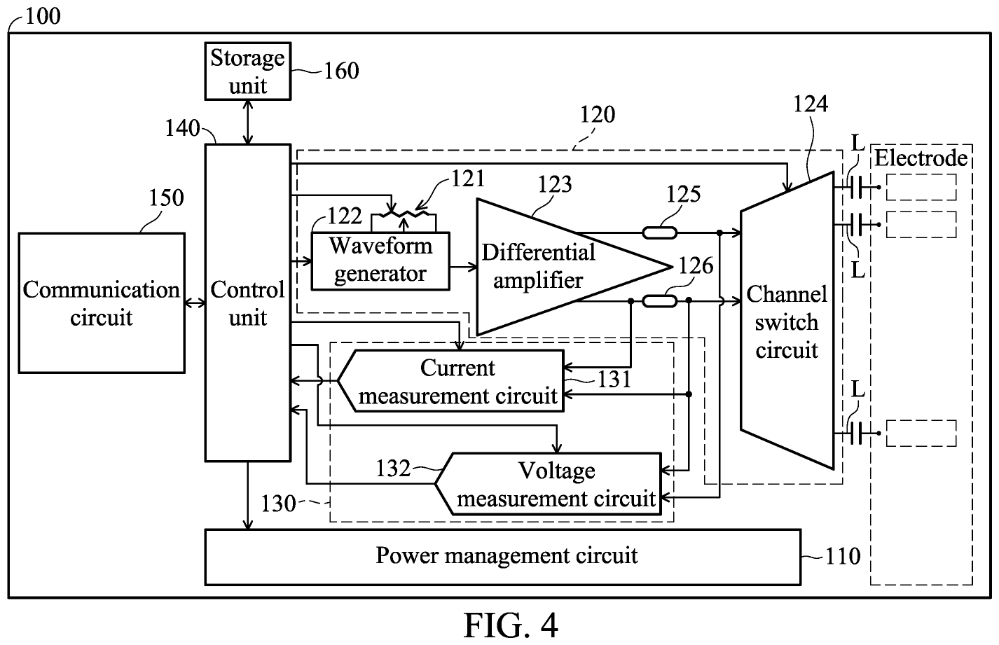

FIG. 5A shows a first set of target energy values according to an embodiment of the present invention. FIG. 5B shows a second set of target energy values according to an embodiment of the present invention. Note that FIGS. 5A and 5B are illustrated merely to explain an embodiment of the present invention, but the present invention is not limited to the first set of target energy values and the second set of target energy values shown in FIGS. 5A and 5B.

As shown in FIG. 5A, the corresponding relations of the various electrical stimulation levels and the various first target energy are stored in the first look-up table, wherein the first set of target energy value may include target energy values X1 to X10. The electrical stimulation levels Level 1 (L1) to Level 10 (L10) correspond to target energy values X1 to X10 respectively, and the target energy values are energy values measured in millijoules. In addition to the electrical stimulation levels L1 to L10 corresponding to the target energy values, they may also correspond to different values for current or voltage. In this embodiment, in the trial phase, when the given electrical stimulation level selected by the user is L6 (i.e. the corresponding given target energy value is X6), the predetermined upper-limit target energy value is X8 and the lower-limit target energy value is X5. The upper-limit target energy value X8 and the given target energy value X6 are separated by one target energy value, and the lower-limit target energy value X5 and the given target energy value X6 are not separated by any target energy value.

In the permanent implantation phase, after obtaining the upper-limit target energy value X8 and the lower-limit target energy value X5, the electrical-stimulation device 100 or the external control device 200 may generate a second set of target energy values according to the upper-limit target energy value X8 and the lower-limit target energy value X5. As shown in FIG. 5B, the second set of target energy values may include target energy values Y1 to Y8, and the target energy values Y1 to Y8 respectively correspond to the electrical stimulation levels L1 to L8 of the external control device 200. In addition, in this embodiment, the minimum target energy value Y1 of the second set of target energy values corresponds to the lower-limit target energy value X5, and the maximum target energy value Y8 corresponds to the upper-limit target energy value X8. In the permanent implantation phase, the electrical-stimulation device 100 and the external control device 200 may perform the electrical stimulation operation according to the second set of target energy values.

According to an embodiment of the present invention, when corresponding to a given electrical stimulation level in the trial phase, the first target energy values include an upper-limit target energy value and a lower-limit target energy value. The upper-limit target energy value and the lower-limit target energy value are brought into the permanent implantation phase, the upper-limit target energy value is the greatest target energy value in the second set of target energy values, and the lower-limit target energy value is the smallest target energy value in the second set of target energy values (as shown in FIG. 5B). As such, it may be ensured that the electrical stimulation in the permanent implantation phase may be performed to the user at an energy intensity near the selected given electrical stimulation level, increasing safety.

According to an embodiment of the present invention, the upper-limit target energy value and the given target energy value are separated by a first number of target energy values, and the lower-limit target energy value and the given target energy value are separated by a second number of target energy values. According to an embodiment of the present invention, the first number (e.g. two) is higher than the second number (e.g. one) (as shown in FIG. 5A). According to another embodiment of the present invention, the first number may be equal to the second number.

According to an embodiment of the present invention, the given target energy value is not included in the second set of target energy values (as shown in FIG. 5B). According to another embodiment of the present invention, the given target energy value may be included in the second set of target energy values.

According to an embodiment of the present invention, the trial phase and the permanent implantation phase may be respectively separated into a non-electrical stimulation phase and an electrical stimulation phase. That is, the trial phase includes a non-electrical stimulation phase and an electrical stimulation phase, and the permanent implantation phase includes a non-electrical stimulation phase and an electrical stimulation phase as well. The non-electrical stimulation phase is the synchronization process when the electrical-stimulation device 100 and the external control device 200 are just started and connected, or after the connection of the electrical-stimulation device 100 and the external control device 200 and before the user starts the electrical stimulation. The electrical stimulation phase is the phase in which the electrical-stimulation device has started to provide the electrical stimulation treatment. Note that the methods described herein for calculating the tissue impedance are equally applicable to both the trial phase and the permanent implantation phase.

According to an embodiment of the present invention, before the electrical-stimulation device 100 performs electrical stimulation on the target area, the control unit 140 of the electrical-stimulation device 100 determines whether the signal quality of the electrical-stimulation signal generated by the electrical-stimulation signal-generating circuit 120 meets the threshold. There is a more detailed explanation in the following paragraphs.

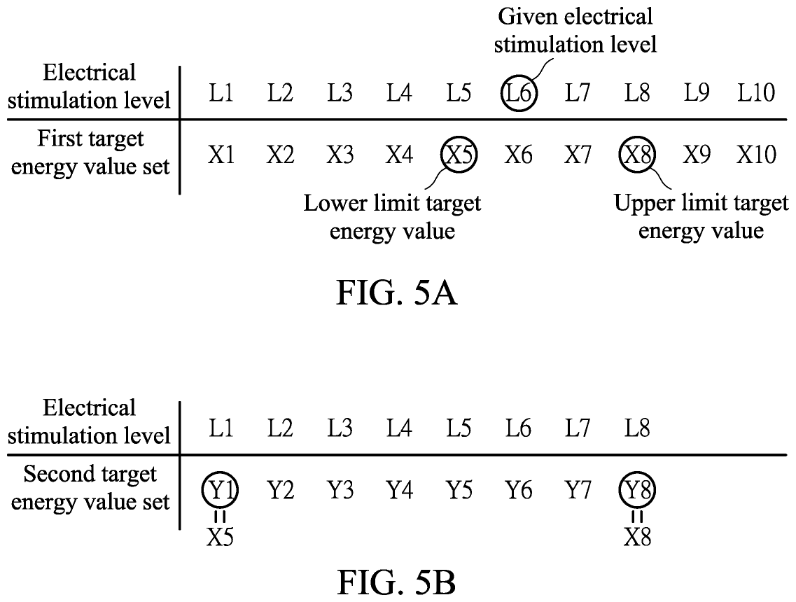

FIG. 6 is a block diagram of the control unit 140 according to an embodiment of the present invention. As shown in FIG. 6, the control unit 140 may include a sampling module 141, a fast Fourier transform calculation module 142, a determination module 143 and a calculation module 144. It should be noted that the block diagram shown in FIG. 6 is only for the convenience of explaining the embodiment of the present invention, but the present invention is not limited to FIG. 6. The control unit 140 may also include other elements. In the embodiment of the present invention, the sampling module 141, the fast Fourier transform calculation module 142, the determination module 143, and the calculation module 144 may be implemented by hardware or software. In addition, according to another embodiment of the present invention, the sampling module 141, the fast Fourier transform calculation module 142, the determination module 143, and the calculation module 144 may also be independent from the control unit 140.

According to an embodiment of the present invention, when the control unit 140 of the electrical-stimulation device 100 determines whether the signal quality of the electrical-stimulation signal generated by the electrical-stimulation signal-generating circuit 120 meets a threshold, the sampling module 141 first samples the electrical-stimulation signal generated by the stimulation signal-generating circuit 120 and sends the electrical-stimulation signal to the fast Fourier transform calculation module 142 to perform a fast Fourier transform. More specifically, the sampling module 141 samples the voltage signal of the electrical-stimulation signal, and the fast Fourier transform calculation module 142 performs a fast Fourier transform on the sampled voltage signal. In addition, the sampling module 141 samples the current signal of the electrical-stimulation signal, and the fast Fourier transform calculation module 142 performs fast Fourier transform on the sampled current signal. In the embodiment of the present invention, the sampling module 141 samples the electrical-stimulation signal during a sampling period, and the sampling period refers to sampling the voltage signal and the current signal of a section of time among the pulse included in each continuous duration T_{d}. That is, sampling the electrical-stimulation signal is equivalent to sampling the pulse signal. According to an embodiment of the present invention, the sampling module 141 first samples the voltage signal of the electrical-stimulation signal (for example, 512 points are sampled), and then samples the current signal of the electrical-stimulation signal (for example, 512 points are sampled). The invention is not limited by the number of samples or the order in which they are sampled.

In an embodiment of the present invention, the sampling module 141 samples each pulse signal in the complex pulse signal. In another embodiment of the present invention, the sampling module 141 samples at least one of the complex pulse signals. For example, the sampling module 141 only samples one pulse signal in every two pulse signals, or the sampling module 141 samples only one pulse signal among every three pulse signals. In one embodiment of the present invention, the unsampled pulse signal may be applied by the data of the pulse signals adjacent to the unsampled pulse signal, but the present invention is not limited thereto. In other words, in an embodiment of the present invention, in a course of electrical stimulation (i.e., the transmission of the first target energy value or the second target energy value to the target area is completed), the sampling module 141 may sample the plurality of pulse signals once or more to obtain a corresponding tissue impedance or a plurality of corresponding tissue impedance values.

The determination module 143 determines whether the signal quality of the electrical-stimulation signal on which the fast Fourier transform was performed meets the threshold. More specifically, the determination module 143 determines whether the first frequency of the voltage signal on which the fast Fourier transform was performed and the second frequency of the current signal on which the fast Fourier transform was performed are equal to a predetermined frequency, so as to determine whether the signal quality of the electrical-stimulation signal meets the threshold. In other words, when the first frequency of the voltage signal on which the fast Fourier transform was performed and the second frequency of the current signal on which the fast Fourier transform was performed are equal to the predetermined frequency, the determination module 143 determines that the signal quality of the electrical-stimulation signal meets the threshold, and when the first frequency of the voltage signal on which the fast Fourier transform was performed and the second frequency of the current signal on which the fast Fourier transform was performed do not equal the predetermined frequency, the determination module 143 determines the signal quality of the electrical-stimulation signal does not meet the threshold. According to an embodiment of the present invention, the predetermined frequency may be between 1 kHz and 1 MHz. According to another embodiment of the present invention, the predetermined frequency may be between 480 kHz and 520 kHz.

According to an embodiment of the present invention, when at least one of the first frequency and the second frequency does not equal the predetermined frequency outside the electrical stimulation stage, the determination module 143 determines whether the voltage of the electrical-stimulation signal meets or exceeds a first predetermined voltage value (for example: 2 volts). When the voltage is lower than the first predetermined voltage value, the determination module 143 increases the voltage of the electrical-stimulation signal by a set amount, and then it re-samples the electrical-stimulation signal. When the voltage meets or exceeds the first predetermined voltage value, the determination module 143 reports that the external control device 200 may not be able to calculate the tissue impedance. According to an embodiment of the present invention, the set amount may be between 0.1 and 0.4 volts, and the first predetermined voltage value may be between 1 and 4 volts, but the invention is not limited thereto. According to an embodiment of the present invention, the initial voltage of the electrical-stimulation signal is also between 0.1 and 0.4 volts. In this embodiment, when the first frequency or the second frequency does not equal the predetermined frequency, the determination module 143 may also increase the value of the counter by one, and determine whether the value of the counter is equal to a predetermined count value. When the value of the counter is equal to the predetermined count value, the determination module 143 reports that the external control device 200 may not be able to calculate the tissue impedance. When the value of the counter is less than the predetermined count value, the determination module 143 determines whether the voltage of the electrical-stimulation signal meets or exceeds a first predetermined voltage value. When the first frequency and the second frequency both equal the predetermined frequency once before the value of the counter reaches the predetermined count value, the counter is reset to zero. According to an embodiment of the present invention, the predetermined count value may be any value between 10 and 30.

According to an embodiment of the present invention, outside the electrical stimulation stage, when the first frequency or the second frequency does not equal the predetermined frequency, the determination module 143 determines whether the average current of the sampled electrical-stimulation signal meets or exceeds a predetermined current value (for example: 2 mA). When the average current is less than the predetermined current value, the determination module 143 increases the voltage of the electrical-stimulation signal by a predetermined value. When the average current meets or exceeds the predetermined current value, the determination module 143 performs the subsequent calculation on the electrical-stimulation signal. According to an embodiment of the present invention, the set amount may be between 0.1 and 0.4 volts, and the first predetermined voltage value may be between 1 and 4 volts, but the invention is not limited thereto. According to an embodiment of the present invention, the initial voltage of the electrical-stimulation signal is also between 0.1 and 0.4 volts.

According to an embodiment of the present invention, during the electrical stimulation stage, when at least one of the first frequency and the second frequency does not equal the predetermined frequency, the determination module 143 re-samples the electrical-stimulation signal, instead using the electrical-stimulation signal sampled this time, or the external control device 200 may know that the electrical-stimulation signal sampled this time is not to be used according to the determination result of the determination module 143. In this embodiment, when at least one of the first frequency and the second frequency does not equal the predetermined frequency, the determination module 143 may use the electrical-stimulation signal that was sampled previously that met the threshold to perform the subsequent electrical stimulation calculation, or the external control device 200 may use the previously sampled electrical-stimulation signal that met the threshold to perform the subsequent electrical stimulation calculation according to the determination result of the determination module 143.

According to an embodiment of the present invention, when the determination module 143 determines that the signal quality of the electrical-stimulation signal meets the threshold, the calculating module 144 calculates the impedance (i.e., the tissue impedance value) corresponding to the sampled electrical-stimulation signal to electrically stimulate a target area. There is a more detailed description below.

According to an embodiment of the present invention, when the determination module 143 determines that the signal quality of the electrical-stimulation signal meets the threshold, the calculation module 144 extracts a first voltage sampling point corresponding to the maximum voltage and a second voltage sampling point corresponding to the minimum voltage in each sampling period, and the maximum voltage and the minimum voltage are subtracted and divided by 2 to generate an average voltage, which may eliminate the background value; it should be noted that as mentioned above, the voltage measurement circuit 132 may raise the voltage to a positive value according to the command of the control unit 140 so as to facilitate the processing of the control unit 140. In addition, when the determination module 143 determines that the signal quality of the electrical-stimulation signal meets the threshold, the calculation module 144 extracts a first current sampling point corresponding to the maximum current and a second current sampling point corresponding to the minimum current in each sampling period, and the maximum current and the minimum current are subtracted and divided by 2 to generate an average current so as to eliminate the background value. After obtaining the average voltage and the average current, the calculation module 144 obtains the aforementioned total impedance according to the average voltage and the average current, and calculates the tissue impedance according to the total impedance. There is a more detailed description below on how to calculate tissue impedance values based on the total impedance. According to another embodiment of the present invention, when the background value is 0, the calculation module 144 may sum the maximum voltage and the minimum voltage and then divide the result by 2 to generate an average voltage, and sum the maximum current and the minimum current and then divide the result by 2, so as to produce the average current.

According to another embodiment of the present invention, when the determination module 143 determines that the signal quality of the electrical-stimulation signal meets the threshold, the sampling module 141 samples all the wave crests and wave troughs of the voltage signal of the electrical-stimulation signal, and the calculation module 144 generates an average voltage according to the values of all the voltage sampling points. For example, the calculation module 144 can average the peaks and valleys included in the 512 sampling points of the voltage signal taken out in each sampling period to generate an average voltage. In addition, the sampling module 141 samples all the wave crests and wave troughs of the current signal of the electrical-stimulation signal, and the calculation module 144 generates an average current according to the values of all the current sampling points. For example, the calculation module 144 may average the wave crests and wave troughs included in the 512 sampling points of the current signal that are extracted in each sampling period to generate an average current. Next, the calculation module 144 obtains a total impedance according to the average voltage and the average current, and calculates the tissue impedance according to the total impedance. There is a more detailed description below on how to calculate tissue impedance values based on total impedance.

According to an embodiment of the present invention, before the electrical stimulation apparatus 100 performs electrical stimulation on the target area, e.g., outside the electrical stimulation stage, the electrical stimulation apparatus 100 calculates the tissue impedance of the target area. According to an embodiment of the present invention, as the electrical-stimulation device 100 shown in FIG. 2A, the electrical-stimulation device 100 may calculate the tissue impedance according to the impedance of the lead and the impedance of the electrical-stimulation device 100. According to another embodiment of the present invention, as the electrical-stimulation device 100 shown in FIG. 2B, the electrical-stimulation device 100 may calculate the tissue impedance according to the impedance of the electrical-stimulation device 100. There is a more detailed description below.

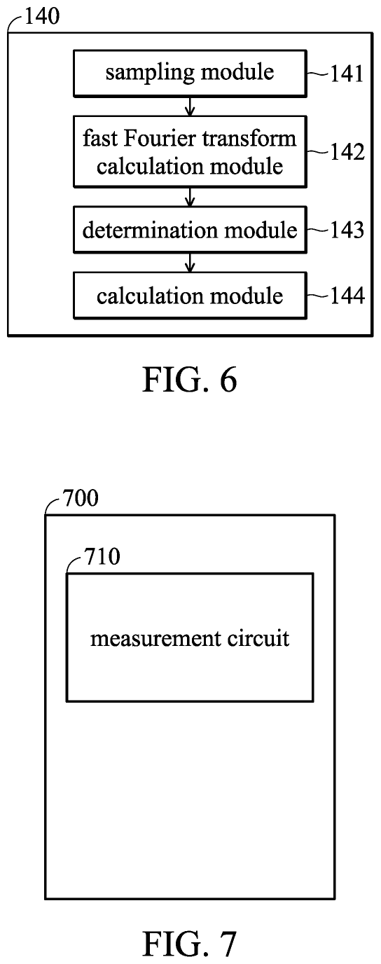

FIG. 7 is a block diagram of an impedance compensation device 700 according to an embodiment of the present invention. As shown in FIG. 7, the impedance compensation device 700 may include a measurement circuit 610, but the present invention is not limited thereto. The measurement circuit 710 may be configured to measure the impedance Z_{Inner} of the electrical-stimulation device 100 and the impedance Z_{Lead} of the lead. According to an embodiment of the present invention, the impedance compensation device 700 (or the measurement circuit 710) may include a related circuit structure as shown in FIG. 4.

According to an embodiment of the present invention, when the measurement circuit 710 is measuring the electrical-stimulation device 100 shown in FIG. 2A, the measurement circuit 710 first provides a high-frequency environment, wherein the frequency (e.g. 500 KHz) equals to the frequency of the electrical-stimulation signal performing the electrical stimulation at the target region. Then, the measurement circuit 710 measures a resistance value R_{Lead}, a capacitance value C_{Lead} and an inductance value L_{Lead} of the lead, and calculates the impedance of the lead Z_{Lead} under a high-frequency signal according to at least one of the resistance value R_{Lead}, the capacitance value C_{Lead}, or the inductance value L_{Lead}. In addition, the measurement circuit 710 measures the resistance value R_{Inner}, the capacitance value C_{Inner}, and the inductance value L_{Inner} of the electrical-stimulation device 100, and calculates the impedance of the electrical-stimulation device 100 Z_{Inner} according to at least one of the resistance value R_{Inner}, the capacitance value C_{Inner}, or the inductance value L_{Inner}. In an embodiment of the present invention, the measurement of the inductance L_{Inner} of the electrical-stimulation device 100 is not required. The measurement circuit 710 writes the calculated impedance of the lead Z_{Lead} and the impedance of the electrical-stimulation device 100 Z_{Inner} to the firmware of the electrical-stimulation device 100.

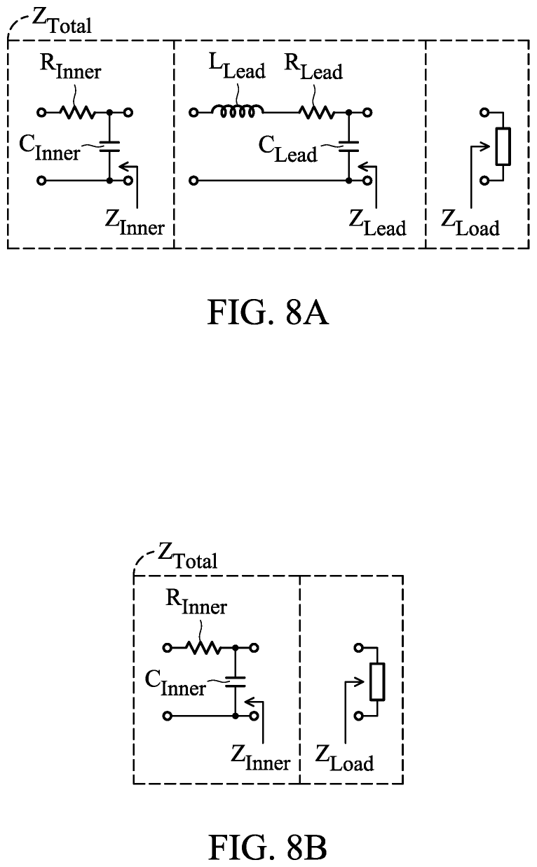

When the electrical-stimulation device 100 is calculating the tissue impedance Z_{Load} at the target region, the electrical-stimulation device 100 may subtract the impedance of the lead Z_{Lead} and the impedance of the electrical-stimulation device 100 Z_{Inner} from the calculated total impedance Z_{Total} to obtain the tissue impedance Z_{Lead} at the target region. As shown in the impedance compensation illustrated in FIG. 8A, Z_{Load}=Z_{Total}−Z_{Inner}Z_{Lead}, but the present invention is not limited thereto. In an embodiment of the present invention, the total impedance Z_{Total} may be calculated by a calculation module 144 through the current measured by the current measurement circuit 131 and the voltage measured by the voltage measurement circuit 132 (i.e. R=V/I). As the calculation of the impedance Z_{Lead} of the lead and the impedance Z_{Inner} of the electrical-stimulation device 100 may refer to the formula Z=R+j(XL−XC), wherein R is the resistance, XL is the inductive reactance and XC is the capacitive reactance, such calculation is well-known by persons skilled in the art and is thus not described in detail herein.

According to another embodiment of the present invention, when the measurement circuit 710 is measuring the electrical-stimulation device 100 shown in FIG. 2B, the measurement circuit 710 first provides a high-frequency environment. The measurement circuit 710 measures a resistance value R_{Inner}, a capacitance value Cₕᵢₗₗₑᵣ and an inductance value L_{Inner} of the electrical-stimulation device 100, and calculates the impedance of the electrical-stimulation device 100 Z_{Inner} according to at least one of the resistance value R_{Inner}, the capacitance value C_{Inner}, or the inductance value L_{Inner}. In an embodiment of the present invention, the measurement of the inductance L_{Inner} of the electrical-stimulation device 100 is not required. The measurement circuit 710 writes the calculated impedance of the electrical-stimulation device 100 Z_{Inner} to the firmware of the electrical-stimulation device 100. When the electrical-stimulation device 100 is calculating the tissue impedance Z_{Load} at the target region, the electrical-stimulation device 100 may subtract the impedance of the electrical-stimulation device 100 Z_{Inner} from the calculated total impedance Z_{Total} to obtain the tissue impedance Z_{Load} at the target region. As shown in the impedance compensation illustrated in FIG. 8B, Z_{Load}=Z_{Total}−Z_{Inner}, but the present invention is not limited thereto.

According to an embodiment of the present invention, the measurement circuit 710 may simulate a high-frequency environment according to an electrical stimulation frequency used by the electrical-stimulation device 100. According to an embodiment of the present invention, the range of the pulse frequency of the high-frequency environment provided by the measurement circuit 710 may be between 1 KHz and 1000 KHz. According to an embodiment of the present invention, the pulse frequency of the high-frequency environment provided by the measurement circuit 710 equals to that of the electrical-stimulation signal.

According to an embodiment of the present invention, the impedance compensation device 700 may be equipped within the external control device 200. According to another embodiment of the present invention, the impedance compensation device 700 may be equipped within the electrical-stimulation device 100. That is, the high-frequency environment may be provided by the electrical-stimulation device 100 or the external control device 200. In addition, according to another embodiment of the present invention, the impedance compensation device 700 may be an independent device (e.g. an impedance analyzer).

According to an embodiment of the present invention, the impedance compensation device 700 may apply to the trial phase (i.e. the electrical-stimulation device 100 is an external electrical-stimulation device with a lead implanted into the human body). According to an embodiment of the present invention, the impedance compensation device 700 may apply to the permanent implantation phase (i.e. the electrical-stimulation device 100 is an implantable electrical-stimulation device, and the electrical-stimulation device 100 may be implanted into the human body along with the lead).

According to an embodiment of the present invention, the impedance compensation device 700 may apply to the electrical-stimulation device 100 before leaving the factory (e.g. in a laboratory or factory). In an embodiment, before the electrical-stimulation device 100 leaves the factory, the impedance compensation device 700 may first calculate the impedance Z_{Lead} of the lead and the impedance Z_{Inner} of the electrical-stimulation device 100, and writes the calculated impedance Z_{Lead} of the lead and the impedance Z_{Inner} of the electrical-stimulation device 100 into the firmware of the electrical-stimulation device 100. In another embodiment, before the electrical-stimulation device 100 leaves the factory, the impedance compensation device 700 may first calculate the impedance Z_{Inner} of the electrical-stimulation device 100, and writes the calculated impedance Z_{Inner} of the electrical-stimulation device 100 into the firmware of the electrical-stimulation device 100. According to an embodiment of the present invention, in the electrical stimulation phase and the non-electrical stimulation phase, the impedance compensation device 700 may perform real-time compensation; that is, each time an electrical-stimulation signal is sent, the impedances Z_{Inner} and Z_{Lead} may be measured.

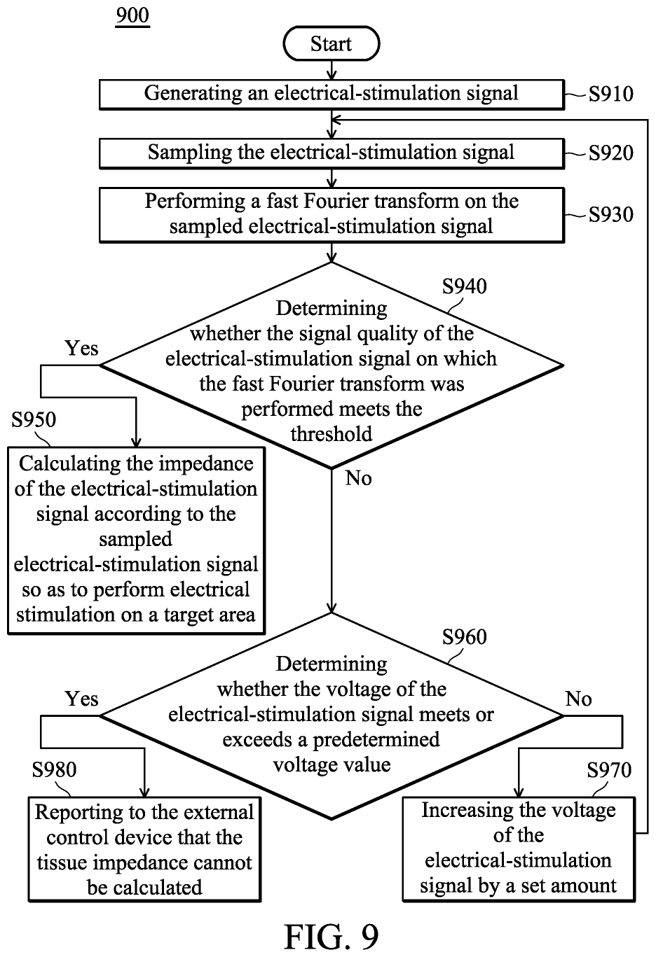

FIG. 9 shows a flow chart 900 of an electrical stimulation method for determining the quality of an electrical-stimulation signal according to an embodiment of the present invention. The flow chart 900 of the electrical stimulation method for determining the quality of an electrical-stimulation signal is applicable to the electrical-stimulation device 100 outside the electrical stimulation stage. As shown in FIG. 9, in Step S910, an electrical-stimulation signal-generating circuit of the electrical-stimulation device 100 generates an electrical-stimulation signal.

In Step S920, a sampling module of the electrical-stimulation device 100 samples the electrical-stimulation signal.

In Step S930, a fast Fourier transform calculation module of the electrical-stimulation device 100 performs a fast Fourier transform on the sampled electrical-stimulation signal.

In step S940, a determination module of the electrical-stimulation device 100 determines whether the signal quality of the electrical-stimulation signal on which the fast Fourier transform was performed meets the threshold.

In Step S950, when the signal quality of the electrical-stimulation signal meets the threshold, a calculation module of the electrical-stimulation device 100 calculates the impedance of the electrical-stimulation signal according to the sampled electrical-stimulation signal so as to perform electrical stimulation on a target area.

In Step S960, when the signal quality of the electrical-stimulation signal does not meet the threshold, the determination module of the electrical-stimulation device 100 determines whether the voltage of the electrical-stimulation signal meets or exceeds a predetermined voltage value.

When the voltage of the electrical-stimulation signal is less than the predetermined voltage value, Step S970 is executed. In Step S970, the determination module of the electrical-stimulation device 100 increases the voltage of the electrical-stimulation signal by a set amount, and Step S920 is executed again to resample the electrical-stimulation signal.

When the voltage of the electrical-stimulation signal meets or exceeds the predetermined voltage value, Step S980 is executed. In Step S980, the determination module of the electrical-stimulation device 100 reports to the external control device 200 that the tissue impedance cannot be calculated.

In this embodiment, before performing Step S960, when the signal quality of the electrical-stimulation signal does not meet the threshold, the determination module of the electrical-stimulation device 100 may first increase the value of the counter by one, and it may determine whether the value of the counter is equal to a predetermined count value. When the value of the counter is equal to the predetermined count value, the determination module of the electrical-stimulation device 100 reports to the external control device 200 that the tissue impedance cannot be calculated. When the value of the counter is less than the predetermined count value, the determination module of the electrical-stimulation device 100 executes Step S960.

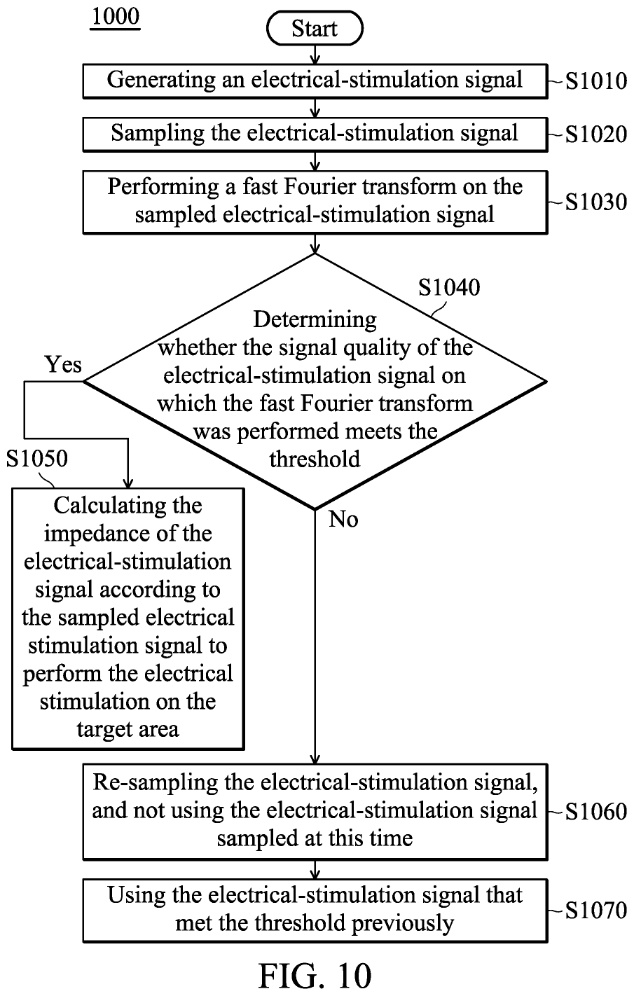

FIG. 10 shows a flow chart 1000 of an electrical stimulation method for determining the quality of an electrical-stimulation signal according to another embodiment of the present invention. The flow chart 1000 of the electrical stimulation method for determining the quality of an electrical-stimulation signal is applicable to the electrical-stimulation device 100 in an electrical stimulation stage. As shown in FIG. 10, in Step S1010, an electrical-stimulation signal-generating circuit of the electrical-stimulation device 100 generates an electrical-stimulation signal.

In Step S1020, a sampling module of the electrical-stimulation device 100 samples the electrical-stimulation signal.

In Step S1030, a fast Fourier transform calculation module of the electrical-stimulation device 100 performs a fast Fourier transform on the sampled electrical-stimulation signal.

In Step S1040, a determination module of the electrical-stimulation device 100 determines whether the signal quality of the electrical-stimulation signal on which the fast Fourier transform was performed meets the threshold.

In Step S1050, when the signal quality of the electrical-stimulation signal meets the aforementioned threshold, the calculation module of the electrical-stimulation device 100 calculates the impedance of the electrical-stimulation signal according to the sampled electrical stimulation signal to determine an impedance value corresponding to the electrical stimulation signal, so as to perform the electrical stimulation on the target area as described above.

In Step S1060, when the signal quality of the electrical-stimulation signal does not meet the threshold, the determination module of the electrical-stimulation device 100 re-samples the electrical-stimulation signal, and does not use the electrical-stimulation signal sampled at this time.

In Step S1070, the determination module of the electrical-stimulation device 100 uses the electrical-stimulation signal that met the threshold previously to perform the subsequent electrical stimulation calculation.

According to an embodiment of the present invention, in Steps S920-S930 and S1020-S1030, the sampling module of the electrical-stimulation device 100 samples the voltage signal of the electrical-stimulation signal, and the fast Fourier transform of the electrical-stimulation device 100 performs fast Fourier transform on the sampled voltage signal. In addition, according to an embodiment of the present invention, in Steps S920-S930 and S1020-S1030, the sampling module of the electrical-stimulation device 100 samples the current signal of the electrical-stimulation signal, and the fast Fourier transform calculation module of the electrical-stimulation device 100 performs a fast Fourier transform on the sampled current signal.

According to an embodiment of the present invention, in Steps S940 and S1040, the determination module of the electrical-stimulation device 100 determines whether the first frequency of the voltage signal on which the fast Fourier transform was performed and the second frequency of the current signal on which the fast Fourier transform was performed the signal equal a predetermined frequency to determine whether the signal quality of the electrical-stimulation signal meets the threshold. According to an embodiment of the present invention, in the aforementioned method for determining the quality of an electrical-stimulation signal, the predetermined frequency may be between 1 kHz and 1 MHz. According to another embodiment of the present invention, in the electrical stimulation method mentioned above for determining the quality of an electrical-stimulation signal, the predetermined frequency may be between 480 kHz and 520 kHz.

According to an embodiment of the present invention, a computer-readable storage medium may store one or more instructions and cooperate with the electrical-stimulation device 100 for performing electrical stimulation. When the computer-readable storage medium stores one or more instructions to be executed by the electrical-stimulation device 100, the electrical-stimulation device 100 may perform a plurality of steps included in the above-described electrical stimulation method of determining the quality of the electrical-stimulation signal.

According to the method for determining the quality of the electrical-stimulation signal proposed by the present invention, the quality of the electrical-stimulation signal may be pre-assessed before performing the electrical stimulation, and the electrical-stimulation signal with poor quality may be excluded.

The ordinal numbers used herein and in the claims, such as “first,” “second” and the like, are used merely for the convenience of description and have no sequential orders among each other.

The steps of the methods and algorithms disclosed herein may be directly applied to hardware, a software module or the combination thereof by executing a processor. A software module (including execution commands and related data) and other data may be stored in a data memory, such as a random access memory (RAM), a flash memory, a read-only memory (ROM), an erasable programmable read-only memory (EPROM), a register, a hard disk drive, a portable hard disk drive, a compact disc read-only memory (CD-ROM), a digital video/versatile disc (DVD), or any other computer-readable storage media format well-known in the art. A storage media may be coupled to a machine device, such as a computer/processor (indicated as a processor herein for the convenience of description), wherein the processor may read information (e.g. a program code) and write information into a storage media. A storage media may be integrated with a processor. An application specific integrated circuit (ASIC) includes a processor and a storage media. A user apparatus includes an ASIC. In other words, a processor and a storage media are included in a user apparatus in a manner that are not directly connected to the user apparatus. In addition, in some embodiments, any product suitable for a computer program includes a readable storage media, wherein a readable storage media includes a program code related to one or more embodiments disclosed herein. In some embodiments, a product of a computer program may include a packaging material.

Various aspects are described in the above description. The teaching herein may be implemented in various ways, and any particular structure or function disclosed in the examples is merely a representative case. According to the teaching herein, it is understood by persons skilled in the art that each aspect of the disclosure herein may be implemented independently, or alternatively, two or more aspects may be implemented in combination.

While the present disclosure has been described in terms of the embodiments, it should be understood that the embodiments are not intended to limit the present disclosure therein. Persons skilled in the art may perform modifications and alterations without departing from the spirit and scope of the present disclosure. Thus, the scope of the invention should be determined by the appended claims.

What is claimed is:

1. An electrical stimulation method for determining quality of an electrical-stimulation signal adapted for use in an electrical-stimulation device for performing an electrical stimulation, comprising:
generating the electrical-stimulation signal;
sampling the electrical-stimulation signal;
performing a fast Fourier transform on the sampled electrical-stimulation signal; and
determining whether a signal quality of the electrical-stimulation signal on which the fast Fourier transform was performed meets a threshold.

2. The electrical stimulation method for determining the quality of an electrical-stimulation signal as defined in claim 1, further comprising:
when the signal quality meets the threshold, calculating an impedance value corresponding to the electrical-stimulation signal according to the sampled electrical-stimulation signal by using the electrical stimulation device so as to perform the electrical stimulation on a target area.

3. The electrical stimulation method for determining the quality of an electrical-stimulation signal as defined in claim 1, further comprising:
sampling a voltage signal of the electrical-stimulation signal and performing the fast Fourier transform on the sampled voltage signal.

4. The electrical stimulation method for determining the quality of an electrical-stimulation signal as defined in claim 1, further comprising:
sampling a current signal of the electrical-stimulation signal and performing the fast Fourier transform on the sampled current signal.

5. The electrical stimulation method for determining the quality of an electrical-stimulation signal as defined in claim 4, further comprising:
determining whether a first frequency of the voltage signal on which the fast Fourier transform was performed and a second frequency of the current signal on which the fast Fourier transform was performed are equal to a predetermined frequency to determine whether the signal quality of the electrical-stimulation signal meets the threshold.

6. The electrical stimulation method for determining the quality of an electrical-stimulation signal as defined in claim 5, wherein the predetermined frequency is between 1 kHz and 1 MHz.

7. The electrical stimulation method for determining the quality of an electrical-stimulation signal as defined in claim 5, wherein the predetermined frequency is between 480 kHz and 520 kHz.

8. The electrical stimulation method for determining the quality of an electrical-stimulation signal as defined in claim 5, wherein outside the electrical stimulation stage, the electrical stimulation method further comprises:
when the first frequency and the second frequency do not equal the predetermined frequency, determining whether a voltage of the electrical-stimulation signal meets or exceeds a predetermined voltage value;
when the voltage is lower than the predetermined voltage value, raising the voltage by a set amount and then resampling the electrical-stimulation signal; and
when the voltage meets or exceeds the predetermined voltage value, reporting to an external control device that tissue impedance cannot be calculated.

9. An electrical-stimulation device configured to perform electrical stimulation, comprising:
an electrical-stimulation signal-generating circuit, configured to generate an electrical-stimulation signal;
a sampling module, configured to sample the electrical-stimulation signal;
a fast Fourier transform calculation module, configured to perform a fast Fourier transform on the sampled electrical-stimulation signal; and
a determination module, configured to determine whether a signal quality of the sampled electrical-stimulation signal on which the fast Fourier transform was performed meets a threshold.

10. The electrical-stimulation device as defined in claim 9, wherein the electrical-stimulation device further comprises a calculation module, wherein when the signal quality meets the threshold, an impedance of the electrical-stimulation signal is calculated according to the sampled electrical-stimulation signal by using the calculation module so as to perform the electrical stimulation on a target area.

11. The electrical-stimulation device as defined in claim 9, wherein the sampling module samples a voltage signal of the electrical-stimulation signal, and the fast Fourier transform calculation module performs the fast Fourier transform on the sampled voltage signal.

12. The electrical-stimulation device as defined in claim 11, wherein the sampling module samples a current signal of the electrical-stimulation signal, and the fast Fourier transform calculation module performs the fast Fourier transform on the sampled current signal.

13. The electrical-stimulation device as defined in claim 12, wherein the determination module determines whether a first frequency of the voltage signal on which the fast Fourier transform was performed and a second frequency of the current signal on which the fast Fourier transform was performed are equal to a predetermined frequency to determine whether the signal quality of the electrical-stimulation signal meets the threshold.

14. The electrical-stimulation device as defined in claim 13, wherein the predetermined frequency is between 1 kHz and 1 MHz.

15. The electrical-stimulation device as defined in claim 13, wherein the predetermined frequency is between 480 kHz and 520 kHz.

16. The electrical-stimulation device as defined in claim 13, wherein outside the electrical stimulation stage, when the first frequency and the second frequency do not equal the predetermined frequency, the determination module determines whether a voltage of the electrical-stimulation signal meets or exceeds a predetermined voltage value, when the voltage is lower than the predetermined voltage value, the determination module raises the voltage by a set amount and then resamples the electrical-stimulation signal, wherein when the voltage meets or exceeds the predetermined voltage value, the determination module reports to an external control device that tissue impedance cannot be calculated.

17. A computer-readable storage medium storing one or more instructions and cooperating with an electrical-stimulation device for performing an electrical stimulation, wherein when the one or more instructions are executed by the electrical-stimulation device, the electrical-stimulation device executes a plurality of steps comprising:
generating an electrical-stimulation signal;
sampling the electrical-stimulation signal;
performing a fast Fourier transform on the sampled electrical-stimulation signal; and
determining whether a signal quality of the electrical-stimulation signal on which the fast Fourier transform was performed meets a threshold.

18. The computer-readable storage medium as defined in claim 17, wherein the steps executed by the electrical-stimulation device further comprise:
when the signal quality meets the threshold, calculating an impedance value corresponding to the electrical-stimulation signal according to the sampled electrical-stimulation signal by using the electrical stimulation device, so as to perform the electrical stimulation on a target area.

19. The computer-readable storage medium as defined in claim 17, wherein the steps executed by the electrical-stimulation device further comprise:
sampling a voltage signal of the electrical-stimulation signal and performing the fast Fourier transform on the sampled voltage signal.

20. The computer-readable storage medium as defined in claim 17, wherein the steps executed by the electrical-stimulation device further comprise:
sampling a current signal of the electrical-stimulation signal and performing the fast Fourier transform on the sampled current signal.

21. The computer-readable storage medium as defined in claim 20, wherein the steps executed by the electrical-stimulation device further comprise:
determining whether a first frequency of the voltage signal on which the fast Fourier transform was performed and a second frequency of the current signal on which the fast Fourier transform was performed are equal to a predetermined frequency to determine whether the signal quality of the electrical-stimulation signal meets the threshold.

22. The computer-readable storage medium as defined in claim 21, wherein the predetermined frequency is between 1 kHz and 1 MHz.

23. The computer-readable storage medium as defined in claim 21, wherein the predetermined frequency is between 480 kHz and 520 kHz.

24. The computer-readable storage medium as defined in claim 21, wherein outside the electrical stimulation stage, the steps executed by the electrical-stimulation device further comprise:
when the first frequency and the second frequency do not equal the predetermined frequency, determining whether a voltage of the electrical-stimulation signal meets or exceeds a predetermined voltage value;
when the voltage is lower than the predetermined voltage value, raising the voltage by a set amount and then resampling the electrical-stimulation signal; and
when the voltage meets or exceeds the predetermined voltage value, reporting to an external control device that tissue impedance cannot be calculated.
